(19) United States
(12) Patent Application Publication
(10) Pub. No.: US 2020/0109455 A1
(43) Pub. Date: Apr. 9, 2020

(54) SYSTEMS AND METHODS FOR PREDICTING CLINICAL RESPONSES TO IMMUNOTHERAPIES

(71) Applicant: THE TRUSTEES OF COLUMBIA UNIVERSITY IN THE CITY OF NEW YORK, New York, NY (US)

(72) Inventors: Raul Rabadan, New York, NY (US); Fabio M. Iwamoto, New York, NY (US); Adam M. Sonabend, Chicago, IL (US); Junfei Zhao, New York, NY (US)

(73) Assignee: THE TRUSTEES OF COLUMBIA UNIVERSITY IN THE CITY OF NEW YORK, New York, NY (US)

(21) Appl. No.: 16/590,017

(22) Filed: Oct. 1, 2019

Related U.S. Application Data
(60) Provisional application No. 62/739,617, filed on Oct. 1, 2018.

(51) Int. Cl. C12Q 1/6886; G16B 20/20; G16B 30/10

(52) U.S. Cl. CPC C12Q 1/6886; G16B 20/20; C12Q 2600/106; C12Q 2600/156; G16B 30/10

(57) ABSTRACT

Systems and methods for predicting a sensitivity of the cancer to an anti-programed cell death 1 (PD-1) immunotherapy are disclosed. The method can comprise determining a presence of at least one mutation in at least one target gene/protein in a sample of the cancer, wherein the target gene can include a PTEN, a PTPN11, and/or a BRAF gene/protein. If the PTPN11 or BRAF gene/protein includes at least one mutation and/or the PTEN gene/protein is a wild type PTEN gene/protein, then the cancer can be predicted to be sensitive to the PD-1 immunotherapy.

### CROSS REFERENCE TO RELATED APPLICATIONS

This application claims priority to U.S. Provisional Application No. 62/739,617, filed on Oct. 1, 2018, the disclosure of which is incorporated by reference herein in its entirety.

### STATEMENT REGARDING FEDERALLY-SPONSORED RESEARCH

This invention was made with government support under grant numbers R01-CA185486, R01-CA179044, U54-CA193313, U54-209997 (RR), and R01-NS103473 awarded by the National Institutes of Health. The government has certain rights in the invention.

### BACKGROUND

Glioblastoma (GBM) is a common primary brain malignancy in adults. Certain immunotherapies with checkpoint inhibitors can have clinical effects in treating tumors.

However, certain clinical trials of immune checkpoints inhibitors have shown limited efficacy for the GBM treatment. For example, a trial involving programmed cell death 1 (PD-1) immune checkpoint inhibitors in recurrent GBM showed that only a small subset of patients (8%) demonstrated objective responses. Clinical effects of anti-PD-1 therapy can be associated with certain gene mutations in tumors across multiple cancer types. The response of GBM patients to PD-1 inhibitor therapies can be unpredictable.

Thus, there is a need for systems and methods to predict and improve clinical responses to immunotherapies.

### SUMMARY

In certain embodiments, the disclosed subject matter provides systems and methods for predicting sensitivity of cancer to an anti-programed cell death 1 (PD-1) immunotherapy. An example method can include determining the presence of at least one mutation in at least one target gene in a sample of cancer. The target gene/protein can include a PTEN, a PTPN11, and/or a BRAF gene/protein. If the PTPN11 or BRAF gene/protein includes at least one mutation and/or the PTEN gene/protein is a wild type PTEN gene/protein, then cancer can be predicted to be sensitive to the PD-1 immunotherapy.

In certain embodiments, the disclosed method can further include determining a presence of at least mutation in the MAPK/ERK pathway components in the sample.

In certain embodiments, the disclosed method can further include determining a PI3K-AKT pathway activity level of the sample.

In certain embodiments, the disclosed method can further include determining the heterozygosity of human leukocyte antigen (HLA) in the sample.

In certain embodiments, the disclosed method can include identifying the clustering of cancer cells and immune cell infiltration. The immune cell can include lymphocytes, neutrophils, macrophages, monocytes, or combinations thereof.

In certain embodiments, the disclosed method can include identifying transcriptomic signatures. The transcriptomic signatures include an immune evasion, a FOXP3 expression, a STAT3 expression, an immunosuppressive response, or combinations thereof.

In certain embodiments, the disclosed method can include providing cancer with a PD-1 inhibitor. The PD-1 inhibitor can include pembrolizumab, nivolumab, or a combination thereof. In non-limiting embodiments, the disclosed method can further include identifying a clonal evolution of cancer before and after the PD-1 inhibitor treatment.

In certain embodiments, the disclosed subject matter provides kits that can be used to practice the disclosed techniques. The kit can include a system including one or more processors; and one or more computer-readable non-transitory storage media coupled to one or more of the processors and comprising instructions operable when executed by one or more of the processors to cause the system to determine a presence of at least one mutation in at least one target gene from a sample of the cancer. In non-limiting embodiments, if the target gene includes a PTEN, a PTPN11, and/or a BRAF gene, and if the PTPN11 or BRAF gene includes at least one mutation and/or the PTEN gene is a wild type PTEN gene, then cancer can be predicted to be sensitive to the PD-1 immunotherapy.

In certain embodiments, the disclosed kit can be further adapted to determine a presence of at least mutation in MAPK/ERK pathway components in the sample. In non-limiting embodiments, the kit can be adapted to determine a PI3K-AKT pathway activity level of the sample. In certain embodiments, the kit can be adapted to determine heterozygosity of HLA in the sample. In certain embodiments, the kit can be adapted to identify clustering of cancer cells, and immune cell infiltration, wherein immune cell includes lymphocytes, neutrophils, macrophages, monocytes, or combinations thereof. In certain embodiments, the kit can be adapted to identify transcriptomic signatures, wherein transcriptomic signatures include an immune evasion, a FOXP3 expression, a STAT3 expression, an immunosuppressive response, or combinations thereof.

In certain embodiments, the kit can further include a PD-1 inhibitor. The PD-1 inhibitor can include pembrolizumab, nivolumab, or a combination thereof. In non-limiting embodiments, the kit can be adapted to identify a clonal evolution of cancer before and after the PD-1 inhibitor treatment.

### BRIEF DESCRIPTION OF THE DRAWINGS

The application file contains at least one drawing executed in color. Copies of this patent with color drawings will be provided by the Office upon request and payment of the necessary fee.

Further features and advantages of the present disclosure will become apparent from the following detailed description taken in conjunction with the accompanying figures showing illustrative embodiments of the present disclosure, in which:

FIG. 1 is a flow diagram illustrating an example sample collection and computational process in accordance with the disclosed subject matter.

FIG. 2 is an image showing brain MRIs of two patients treated with nivolumab.

FIG. 3A is a graph illustrating univariate survival analysis. FIG. 3B is a graph showing a Kaplan-Meier curve comparing overall survival of patients who responded to anti-PD-1 therapy to those that did not respond.

FIG. 4A is a diagram showing clinical and genetic profiles. FIG. 4B is a graph illustrating the enrichment of BRAF/PTPN11 and PTEN mutations in tumors from responders and non-responders. FIG. 4C is a diagram showing locations of identified mutations within the PTEN protein.

FIG. 5A is a diagram showing evolutionary trees of 5 patients (2 Non-Responders & 3 Responders) evaluated by whole-exome sequencing. FIG. 5B is a diagram illustrating tumor evolution models which can characterize Non-Responders and Responders in accordance with the disclosed subject matter. FIG. 5C is a graph showing a variant allele frequency of protein-coding mutations before and after immunotherapy.

FIG. 6A is heatmap showing the top gene sets differentially enriched in Responders versus Non-Responders prior to (left) and after immunotherapy (right). FIG. 6B is a graph showing T-cell clonal diversity before and after immunotherapy. FIG. 6C is a graph illustrating a single-cell RNA-Seq for identifying a cluster of CD44-expressing tumor cells. FIG. 6D is a heatmap showing the associations between PTEN mutation and immune cell enrichment.

FIGS. 7A-B are representative multispectral mages (MSI) showing DAPI (nuclei, blue), SOX2 (tumor, red), CD68 (microglia/macrophages, green), HLA-DR (activation marker, orange), CD3 (T cells, cyan), PDL-1 (immune suppression, yellow), and CD8 (CTLs, magenta), in a non-responder (7A) and a responder (7B). FIG. 7C is a graph illustrating cellular proportions for identified cell types before and after immunotherapy. FIG. 7D is a graph showing pair correlation functions which compare the degree of clustering of cells as a function of radius, for macrophages in PTEN-wildtype patients (above) and for tumor cells prior to immunotherapy (below).

FIG. 8 is a diagram of the data modalities available across the 50-patient cohort.

FIG. 9 is a graph showing a Kaplan-Meier curve comparing overall survival from diagnosis of patients who responded to anti-PD-1 therapy to those that did not respond.

FIG. 10A is a graph showing a mutation burden by response group. FIG. 10B is a graph illustrating a ratio of sub-clonal to clonal mutations, as estimated by ABSOLUTE, by response group. FIG. 10C is a graph showing a tumor purity, as estimated by ABSOLUTE, by response group. FIG. 10D is a graph showing an aneuploidy score analysis of non-responder vs responder.

FIG. 11 is graph illustrating GSEA enrichment score of gene set KIM_PTEN_TARGETS_UP for non-responders vs responders.

FIG. 12 is a graph showing a boxplot of CD274/PDL1 mRNA expression in responders vs. non-responders.

FIG. 13 is a graph showing survival curves versus HLA homozygosity in the TCGA background.

FIG. 14 is an image showing clonal diversity of lymphocytes before and after immunotherapy.

FIG. 15 is a graph illustrating B-cell clonal diversity before and after immunotherapy.

FIG. 16 is a map showing expression subtyping of tumors from 9 patients (pre- & post-treatment) into proneural, mesenchymal, and classical subtypes.

FIG. 17 is a graph showing GSEA enrichment plots (responder vs non-responder) of two regulatory T (Treg) cells related gene sets.

FIG. 18A is a graph showing cells associated with the regulatory T-cells signature. FIG. 18B is a graph illustrating tumors associated with the regulatory T-cells signature.

FIG. 19 is a diagram showing a topological data analysis of single-cell RNA-seq data from two PTEN wildtype tumors.

FIG. 20 is a graph illustrating PTEN mutated GBM tumors which have lower tumor purity compared to PTEN wild-type tumors.

FIG. 21 is a graph showing mutation loads of Non-responsive and responsive patients before and after treatment.

FIG. 22 is a heat map showing alternations of genes in Responders and Non-Responders.

Throughout the figures, the same reference numerals and characters, unless otherwise stated, are used to denote like features, elements, components or portions of the illustrated embodiments. Moreover, while the present disclosure will now be described in detail with reference to the figures, it is done so in connection with the illustrative embodiments.

### DETAILED DESCRIPTION

The disclosed subject matter provides techniques for predicting the sensitivity of cancer to an anti-programed cell death 1 (PD-1) immunotherapy. The disclosed subject matter can provide biomarkers for patients with cancer or tumor in need of determining if a patient can benefit from the treatment with immunotherapy.

In certain embodiments, the disclosed subject matter can provide a method for predicting the sensitivity of cancer to PD-1 immunotherapy. The method can include determining a presence of at least one mutation in at least one target gene/protein. In non-limiting embodiments, the target gene/protein can include MAPK/ERK pathway components. For example, a presence of at least one mutation in BRAF and/or Tyrosine-protein phosphatase non-receptor type 11 (PTPN11) gene/protein can be determined by the disclosed techniques.

Such mutations can be used to predict the sensitivity of cancer to immunotherapy. For example, if the PTPN11 or BRAF gene/protein includes at least one mutation, then cancer can be predicted to be sensitive to immunotherapy (e.g., PD-1 immunotherapy). MAPK/ERK pathway components can be more frequently mutated in the responsive tumors (e.g., glioblastomas and melanoma) than the non-responsive tumors. Such alternations of MAPK/ERK pathway components in tumor cells can be recognized by cytotoxic T-cells which can affect a PD1-inhibitor therapy. In some embodiments, MAPK targeted therapy can be combined with the PD-1 immunotherapy for treating cancers.

In certain embodiments, the target gene can include any genes which can affect functions of phosphatase and tensin homolog (PTEN). For example, a presence of at least one mutation which can affect the function of PTEN gene/protein in cancer can be determined by the disclosed technique. Such mutations can be used to predict the sensitivity of cancer to immunotherapy. For example, if no PTEN loss of function mutations are identified (e.g., Wild type PTEN), then cancer can be predicted to be sensitive to immunotherapy (e.g., PD-1 immunotherapy). PTEN can be more frequently mutated in the non-responsive tumors than the responsive tumor. Loss of PTEN in tumor cells can increase the expression of immunosuppressive cytokines resulting in decreased T-cell infiltration in tumors and inhibited autophagy. Such effects can decrease T-cell-mediated cell death. In noon-limiting embodiments, tumor-specific T-cells can lysate PTEN wild type glioma cells more efficiently than those expressing mutant PTEN. In some embodiments, the level of PI3K-AKT pathway activity can be determined by the disclosed techniques. For example, higher level of PI3K-AKT pathway activity among PTEN mutant non-responsive tumors can be detected than responsive tumors.

In certain embodiments, the disclosed method can further include identifying human leukocyte antigen (HLA) heterozygosity. HLA heterozygosity can correlate with anti-PD-1 response. HLA in non-responsive cancer can be more frequently homozygous for at least one HLA-I locus (A, B or C) than responsive cancer. For example, about 25% of homozygosity at HLA-I loci (e.g., 117/394) suggests that the HLA-I homozygosity can be enriched in non-responders.

In certain embodiments, the disclosed method can further include identifying the structure of the tumor microenvironment. The structure of the tumor microenvironment can include immune cell infiltration and clustering of the tumor cell. The immune cells can include lymphocytes (T, B, and NK cells), neutrophils, macrophages (e.g., tumor-associated macrophages (TAMs)), and monocytes. In some embodiments, immune cell infiltration can correlate with response to anti-PD-1 treatment. For example, PTEN-mutated tumors can have higher overall levels of macrophage infiltration then PTEN-wildtype tumors. In non-limiting embodiments, the density of T cells in PTEN-wildtype tumor can increase after the immunotherapy treatment samples.

In certain embodiments, the disclosed method can further include identifying enriched transcriptomic signatures. The transcriptomic signatures can include an immune evasion, a FOXP3 expression, a STAT3 expression, and/or an immunosuppressive signature. For example, immune signatures can be up-regulated in non-responsive tumors compared to responsive tumors.

In non-limiting embodiments, the disclosed subject matter provides techniques to identify PTEN gene/protein which can affect the formation of the tumor immune microenvironment and regulate the immune signatures. Comparing PTEN-mutated samples with PTEN wild-type samples, PTEN mutation can correlate with the FOXP3-related transcriptional signature, and with lower tumor purity. In some embodiments, PTEN mutations can be associated with higher level of macrophages, microglia, and neutrophils in the tumor microenvironment than PTEN-wild type tumors. For example, macrophages (e.g., TAMs) can release growth factors and cytokines in response to factors produced by cancer cells. The macrophages can facilitate tumor proliferation, survival and migration altering patients' responses to anti-PD-1 therapy.

In certain embodiments, the disclosed method can further include providing immunotherapy to the tumor. For example, a patent with cancer (e.g., GBM, melanoma, gastrointestinal cancer and glioma) can be treated with checkpoint inhibitors. The checkpoint inhibitors can include PD-1 inhibitors (e.g., pembrolizumab or nivolumab). In non-limiting embodiments, the immunotherapy can be provided with other therapies. For example, PD-1 checkpoint inhibitors can be combined with MAPK targeted therapies for treating multiple cancers.

In certain embodiments, the disclosed method can further include identifying a clonal evolution of tumors. The immune system can function to select tumor variants with reduced immunogenicity, thereby providing tumors with a mechanism to escape immunologic detection and elimination. For example, after the anti-PD-1 immunotherapy treatments, the treated tumors can obtain such immune-evasive features. The disclosed subject matter provides techniques to detect such immune-evasive features by identifying a clonal evolution of tumors. For example, an evolutionary tree of a tumor can be constructed using the number of mutations exclusive to or in common with each tumor sample.

The tumors from non-responders and responders can exhibit different patterns of evolution. Non-responsive tumors can have higher fraction of mutations exclusive to post-immunotherapy tumors compared to the pre-anti-PD-1 treatment case. Responsive tumors can have specific alterations and evolutionary patterns which are associated with treatment. For example, expression level of missense mutations (e.g., MYPN R409H, UBQLN3 R159W, CYP27B1 G194E, FNIP1 T409M, TCF12 A605S) can be altered after the anti-PD-1 treatment.

In certain embodiments, the method can include identifying clonal evolution of lymphocytes before and after immunotherapy. For example, lymphocyte clonal diversity can be assessed by identifying TCR and immunoglobulin RNA sequences. Clonal diversity can be accessed via immunoglobulin reads and/or Shannon entropy, an information theory measure of randomness. Non-responsive tumors can have a greater increase in clonal diversity, and immunoglobulin reads among T cells after immunotherapy compared to responsive tumors.

In certain embodiments, the disclosed subject matter provides kits that can be used to practice the disclosed techniques. For example, the kit can include a system including one or more processors and one or more computer-readable non-transitory storage media coupled to one or more of the processors and comprising instructions operable when executed by one or more of the processors to cause the system to determine a presence of at least one mutation in at least one target gene from a sample of the cancer. In non-limiting embodiments, if the target gene includes a PTEN, a PTPN11, and/or a BRAF gene, and if the PTPN11 or BRAF gene includes at least one mutation and/or the PTEN gene is a wild type PTEN gene, then cancer can be predicted to be sensitive to the PD-1 immunotherapy. In certain embodiments, the disclosed kit can be further adapted to determine a presence of at least mutation in MAPK/ERK pathway components in the sample. In non-limiting embodiments, the kit can be adapted to determine a PI3K-AKT pathway activity level of the sample. In certain embodiments, the kit can be adapted to determine heterozygosity of HLA in the sample. In certain embodiments, the kit can be adapted to identify clustering of cancer cells, and immune cell infiltration, wherein immune cell includes lymphocytes, neutrophils, macrophages, monocytes, or combinations thereof. In certain embodiments, the kit can be adapted to identify transcriptomic signatures, wherein transcriptomic signatures include an immune evasion, a FOXP3 expression, a STAT3 expression, an immunosuppressive response, or combinations thereof.

In certain embodiments, the kit can further include a PD-1 inhibitor. The PD-1 inhibitor can include pembrolizumab, nivolumab, or a combination thereof. In non-limiting embodiments, the kit can be adapted to identify a clonal evolution of cancer before and after the PD-1 inhibitor treatment.

In certain embodiments, the disclosed subject matter provides techniques to identify patients (i.e., responder) who can respond to anti-PD-1 immunotherapy with an improved survival rate. The disclosed techniques can also identify responsive and non-responsive tumors by assessing PTEN mutations, microenvironments, MAPK/ERK pathway components, HLA heterozygosity, and/or transcriptomic signatures. The disclosed subject matter also provides techniques to identify effects of immunotherapy which can promote tumors in escaping immune surveillance by assessing a clonal evolution of tumors and lymphocytes. Patterns in the changes of the clonal composition of lymphocytes/tumors between responders and non-responders can be identified by the disclosed techniques. In non-limiting embodiments, the disclosed subject matter provides techniques which can inform therapeutic options and identify mechanisms of resistance to immunotherapies in these tumors.

The following Example is offered to more fully illustrate the disclosure but is not to be construed as limiting the scope thereof.

### Example 1: Longitudinal Genomic Study of Response to Anti-PD-1 Immunotherapy in GBM

The current standard of care for newly-diagnosed glioblastoma has limited efficacy, with a median overall survival of approximately 16-20 months. Given the poor prognosis and limited treatment options for patients with glioblastoma, there has been considerable interest in investigating the efficacy of immunotherapy in this disease. Certain immunotherapies with checkpoint inhibitors can be used for treating a variety of tumors, including advanced melanoma, non-small-cell lung cancer, and Hodgkin's lymphoma, among others. For example, programmed cell death 1 (PD1) immune checkpoint inhibitors can be used for patients with recurrent glioblastoma.

Response to anti-PD-1 therapy can be associated with mutations in tumors across multiple cancer types. Levels of T-cell infiltration in the tumor microenvironment can also be associated with the likelihood of response. Compared to melanomas or non-small cell lung cancer, GBM can harbor a lower burden of somatic mutations and a more immunosuppressed tumor microenvironment. Multiple mechanisms can lead to an immunosuppressive microenvironment in GBM, including restricted lymphocyte trafficking due to the blood-brain barrier, the inhibition of T-cell proliferation and effector responses, the exhaustion and apoptosis of cytotoxic lymphocytes, the production of immunosuppressive cytokines, the activation of FOXP3+ regulatory T-cells (Tregs), the recruitment of myeloid-derived immunosuppressive cells, and the polarization of macrophages. T-cell exhaustion and apoptosis can be modulated by PD-1 ligands (PD-L1/2) expressed by tumor cells: upon binding PD1 on the surface of cytotoxic T-cells, the T-cells can become incapable of eliciting effective anti-tumor responses. PD-1 inhibitor therapy can impair this immune checkpoint and enhances the anti-tumor immune response. Given the uncommon and unpredictable response of GBM patients to PD-1 inhibitor therapies and the variability of immunosuppressive features across these tumors, the disclosed techniques were used to provide to predict responses to immunotherapy.

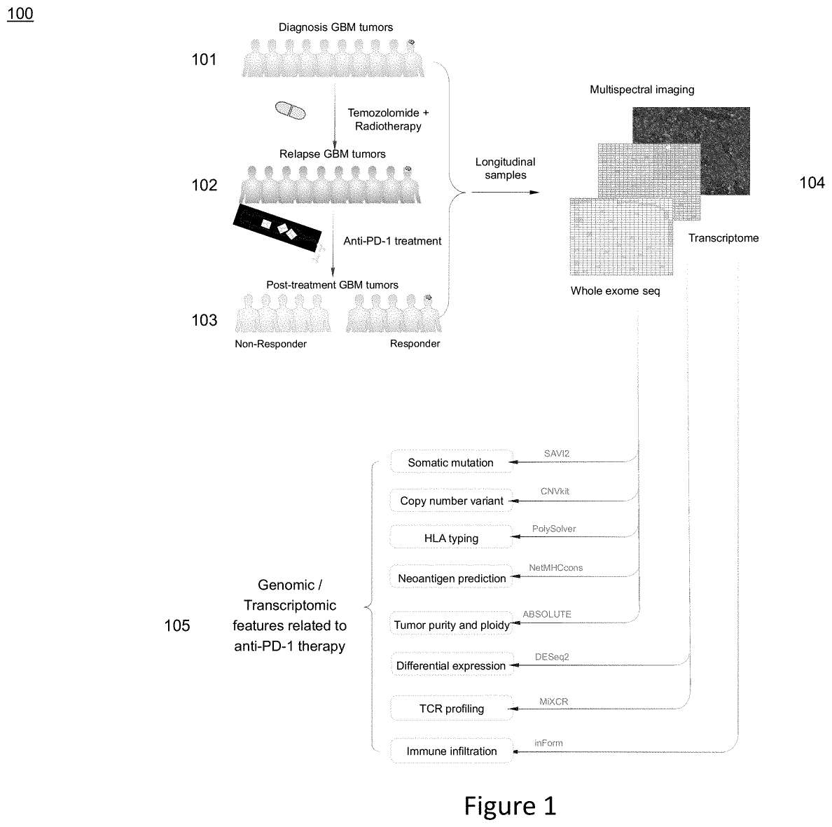

50 patients were profiled (101) across a variety of time points 102 and 103, including the collection of DNA, RNA, tissue imaging, and clinical data 104 (FIG. 1). Genomic and stromal features associated with clinical outcomes were observed to gain insight into the underlying mechanisms of immunotherapy response 105.

Sequencing and mapping: reads for these samples were mapped by BWA to the hg19 human genome assembly with default parameters. All mapped reads were then marked for duplicates by Picard to eliminate potential duplications. The Picard is a set of software to mify high-throughput sequencing data and formats.

Somatic mutations: to identify somatic mutations from whole-exome sequencing data for samples from patients with GBM, the variant-calling software SAVI2 (statistical algorithm for variant frequency identification) was applied, which can be based on an empirical Bayesian method. A list of variant candidates was generated by eliminating positions without variant reads, positions with low sequencing depth, positions that were biased for one strand, and positions that contained only low-quality reads. Then, the numbers of high-quality reads for forward-strand reference alleles, reverse-strand reference alleles, forward-strand non-reference alleles, and reverse-stand non-reference alleles were calculated at the remaining candidate positions to build the prior and the posterior distribution of mutation allele fraction. Finally, somatic mutations were identified based on the posterior distribution of differences in mutation allele fraction between normal and tumor samples. Statistical Algorithm for Variant Frequency Identification 2 (SAVI2) was able to assess mutations by simultaneously considering multiple tumor samples, as well as their corresponding RNA samples, if available.

Analysis of copy number changes: CNVkit, a command-line software to visualize copy number from DNA sequencing data, was used to detect copy number changes from whole-exome sequencing data.

Gene fusion detection: ChimeraScan, a software for identifying chimeric transcription in sequencing data, was used to generate the starting set of gene fusion candidates. To reduce the false-positive rate and nominate potential driver events, the Pegasus annotation and prediction pipeline were applied. Pegasus can provide a common interface for various gene fusion detection tools, reconstruction of novel fusion proteins, reading-frame-aware annotation of preserved/lost functional domains, and data-driven classification of oncogenic potential. Pegasus can streamlines the search for oncogenic gene fusions, bridging the gap between raw RNA-Seq data and a final, tractable list of candidates for experimental validation. The entire fusion sequence on the basis of breakpoint coordinates was reconstructed, and a driver score was assigned to each candidate fusion via a machine learning model trained largely on GBM data.

Gene expression analysis: paired-end transcriptome reads were processed using the spliced transcripts alignments to a reference (STAR) aligner based on the Ensembl GRCh37 human genome assembly with default parameters. Normalized gene expression values were calculated by featureCounts (i.e., a software program for counting reads to genomic features such as genes, exons, promoters and genomic bins) as RPKM. The single sample gene set enrichment analysis (ssGSEA) was performed using gene set variation analysis (GSVA) of R package. Then differentially-enriched gene sets between the Responders and Non-Responders were defined by an effect size of GSVA score differences being greater than 0.8 and a t-test p-value of less than 0.01.

HLA typing and neoantigen prediction: HLA typing for each sample was performed using the POLYSOLVER algorithm, a software for HLA typing based on whole exome sequencing data. The personalized variant antigens by cancer sequencing (pVAC-Seq) pipeline was used with the “NetMHCcons” binding strength predictor to identify neoantigens. NetMHCcons integrates “NetMHC”, “NetMHCpan”, and “PickPocket” to give improved predictions. The variant effect predictor from Ensembl was used to annotate variants for downstream processing by pVAC-Seq. For each single-residue missense alteration, MHC binding affinity was predicted for all the wild-type and mutant peptides of 8, 9, 10, and 11 amino acids in length. The mutant peptide with the strongest binding affinity was kept for further analysis.

Single-cell data analysis: single-cell transcriptional profiles were obtained from 9,000 cells over three samples. GSEA was used to assess enrichment of transcriptomic signatures among the samples. Topological representations of cellular expression were constructed with Mapper (Ayasdi Inc), outputting a network where nodes represent sets of cells with similar characteristics. RGB values were computed for each node in proportion to its composition of Ki67+ tumor cells, microglia, and CD44+ tumor cells, respectively.

Tumor purity estimation and cellular fraction: “ABSOLUTE” was used to infer tumor purity and ploidy for each whole-exome sequencing sample by integrating mutational allele frequencies and copy number calls.

Quantitative multiplex Immunofluorescence (qmIF) analysis: formalin-fixed, paraffin-embedded (FFPE) tumor samples were collected for each sample and Hematoxylin. Eosin (“H&E”) slides were reviewed by a neuropathologist (PC) to confirm the presence of tumor. Opal multiplex staining was performed on FFPE immunoblank slides for CD3 (T cells), CD8 (cytotoxic T lymphocytes (CTLs)), CD68 (microglia/macrophages), HLA-DR (activation marker), PD-L1 (immunosuppression marker), and SOX2 (tumor marker). Images were acquired using Vectra™ (PerkinElmer) for whole slide scanning, and multispectral images (MSI) were acquired for all areas with at least 99% tissue, using inForm™ software (PerkinElmer) to unmix and remove autofluorescence. MSIs were analyzed using inForm™ software and R to evaluate density of immune phenotypes within the tumor microenvironment.

Spatial Analysis: phenotyped immunofluorescence data was processed into pair correlation functions (PCFs) using “the spatstat R package.” Inhomogeneous PCFs were calculated up to a radius of 50 microns for Tumor and CD68+ cells provided that there was a minimum of 20 cells of that type in the sample. The isotropic edge correction and a normalization power of 2 were used. The area under the curve for each PCF was used as a summary statistic for quantifying clustering, and plots represent the point-wise median PCF across samples with 95% confidence intervals obtained via bootstrapping.

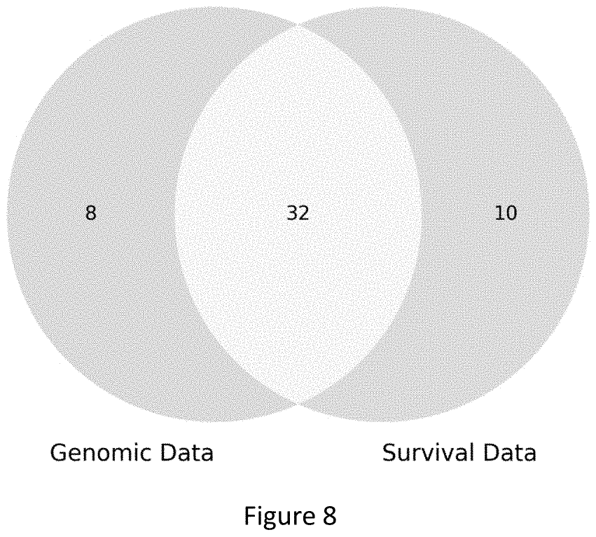

Response to Anti-PD-1 Immunotherapy Correlates with Improved Post-treatment Patient Survival: a retrospective series of adult GBM patients who were treated with PD1 inhibitors pembrolizumab or nivolumab upon recurrence was compiled (n=50). All patients were treated with the standard therapy of temozolomide and radiation prior to the administration of PD-1 inhibitors. Patients were excluded for which there were no pre-immunotherapy specimens (either at diagnosis or after standard therapy recurrence) available. The distribution of available data modalities across the patient cohort is listed in FIG. 8.

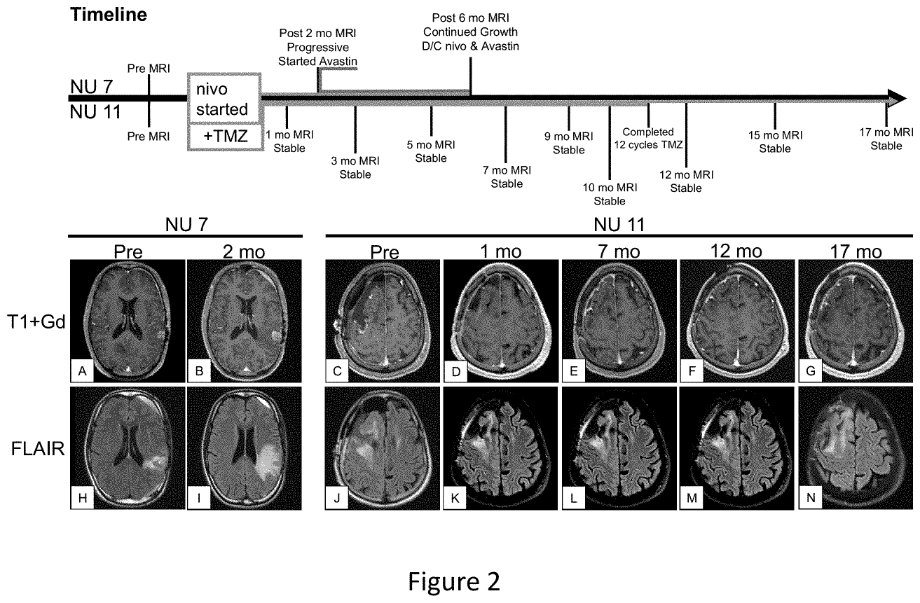

Patients were classified as responders if they met at least one of the following two criteria: 1) Tissue sampled during surgery after PD1 inhibitor therapy grossly showed only an inflammatory response and very few to no tumor cells (as associated with pseudo-progression). 2) Tumor volumes, as seen from MRI were either stable or shrinking continually over at least six months. FIG. 2 shows brain MRIs of two patients treated with nivolumab with their corresponding relative timelines. Patient NU 7 showed progression after two months of nivolumab as measured by the RANO criteria. Meanwhile, patient NU 11 showed stable disease without progression after 17 months.

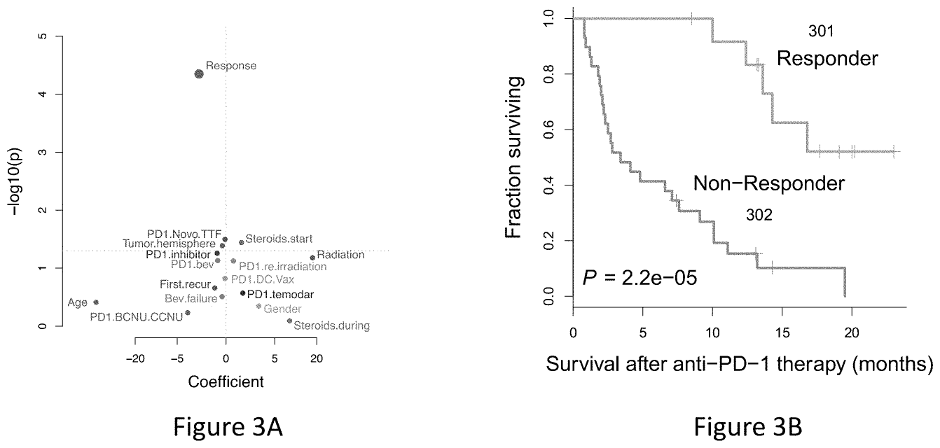

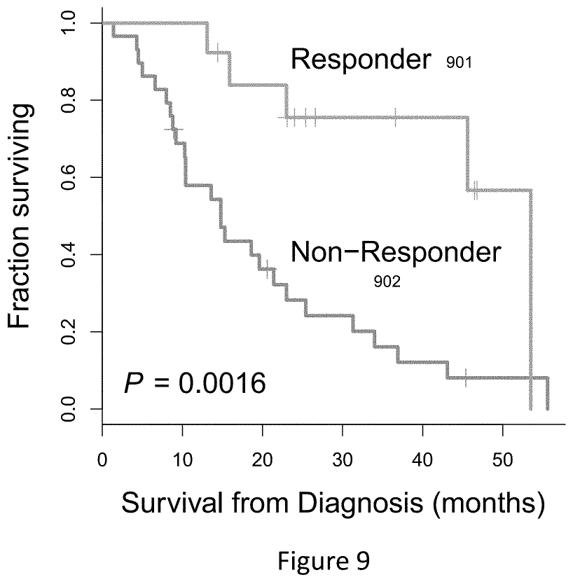

Demographic and clinical characteristics, including response pattern, age at treatment initiation, gender, and choice of PD-1 inhibitor were evaluated in univariate survival analysis (FIG. 3A). One covariate, response to the PD-1 inhibitor, was found to be significantly associated with overall survival as measured from the initiation of immunotherapy: patients who showed a responsive pattern to anti-PD-1 immunotherapy had a median survival of 15.5 months compared to the 5.7 months of non-responsive patients 302 (p=2.2e−5, log-rank test) (FIG. 3B). Similarly, survival, as measured from initial diagnosis, was also increased in responders 901 (p=1.6e−3, log-rank test, FIG. 9). However, there was no significant difference in the time spanning between initial diagnosis and the start of anti-PD1 treatment between the two groups (p=0.96, Wilcoxon rank-sum test).

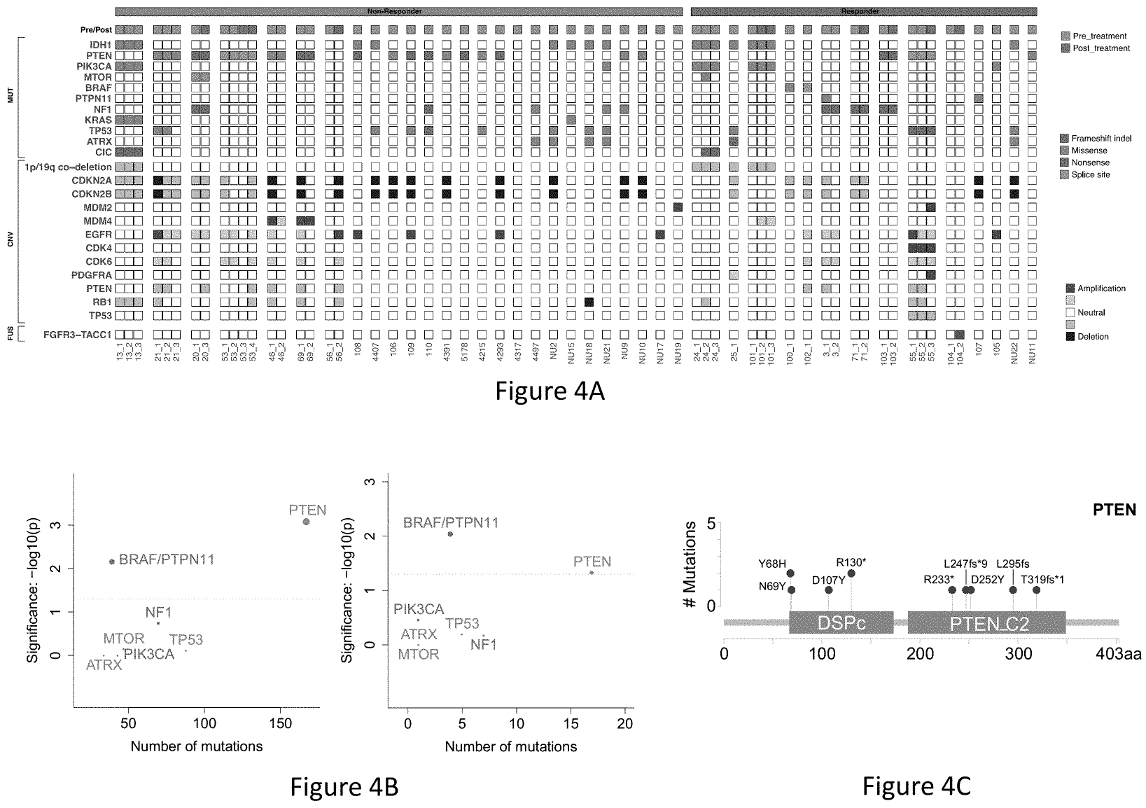

Enrichment of PTEN Mutations in Anti-PD-1 Non-Responsive GBM. 58 whole exomes and 38 transcriptomes from longitudinal tumor-matched blood normal samples for 17 patients with GBM who received anti-PD-1 immunotherapy were analyzed. Paired samples with timepoints, both pre- and post-anti-PD-1 treatment were available for seven patients. The results from a cancer gene panel for 23 patients were also incorporated (FIG. 4A).

On average, 100-fold exome-wide target coverage was achieved for all of the sequenced tumor samples and 60-fold for matched blood normal samples. To identify somatic single-nucleotide variants (SNVs) as well as short insertions and deletions (indels), the variant calling software SAVI2 was used. Only variants with a mutant allele frequency of 5% or greater were included for further analysis. A median of 47 non-synonymous somatic mutations in the 33 tumors was observed (with a range from 14 to 83, which is the pattern typically observed in GBM19).

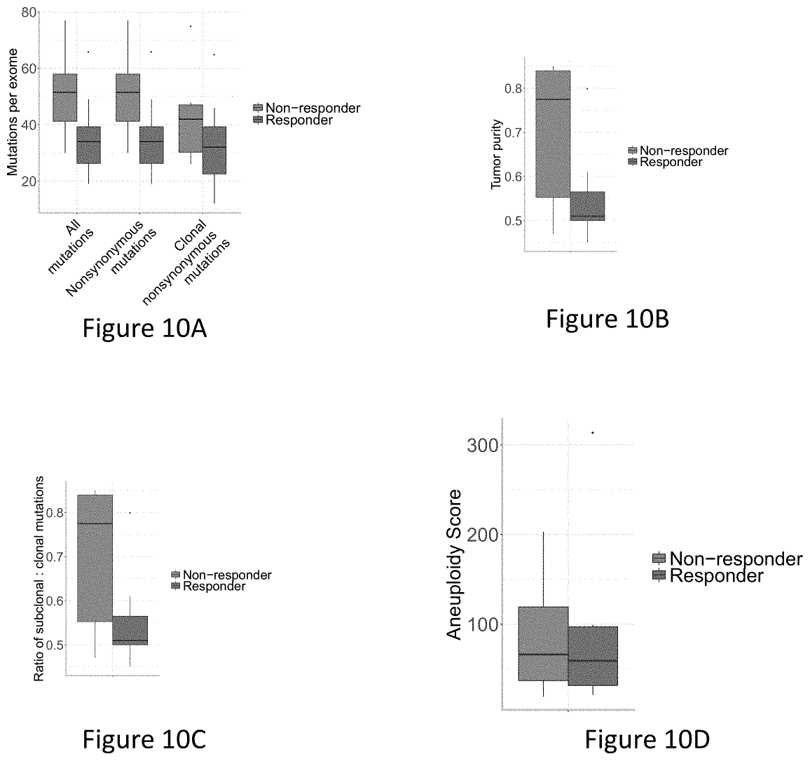

No more non-synonymous single nucleotide variants (nsSNVs) in the responsive tumors was found compared to the non-responsive baseline tumors in the disclosed cohort (FIG. 10). This comparison was based on the pre-treatment samples from the 1st surgery for each patient, with a median nsSNV count of 40 for non-responders and 26 for responders (p=0.11, Wilcoxon rank-sum test). A statistically non-significant trend was observed between response and aneuploidy (p=0.88, t-test, FIG. 10). Similarly, human leukocyte antigen class I (HLA-I) neoepitope load predictions yielded similar patterns for the two groups (median of 37 in non-responders and 31 in responders, p=0.65, Wilcoxon rank-sum test). Additionally, the tumor purity of each sample was estimated using ABSOLUTE. This comparison showed that there was no significant difference in tumor purity between these two groups (median of 0.41 in non-responders and 0.38 in responders, p=0.19, Wilcoxon rank-sum test).

Mutations (nsSNVs and indels) that were enriched in either responsive or non-responsive tumors were identified. In total, 11 IDH1 R132G/H mutated tumors were identified, of which 4 were found in responders and 7 in non-responders. Focusing on the remaining 29 IDH1 wild-type tumors, 14 PTEN mutations were found among the 19 non-responders, but only 3 among the 10 responders (FIG. 4B). Considering that the background PTEN mutation rate is around 33% (154 of 458 tumors in IDH1 wild-type glioblastomas from TCGA23), PTEN was more frequently mutated in the non-responsive tumors than expected (Fisher p=0.0078, odds ratio=5.5, FIG. 4B, left). Similar results were obtained when comparing PTEN status within the cohort itself (Fisher p=0.046, odds ratio=6.0, FIG. 4B, right).

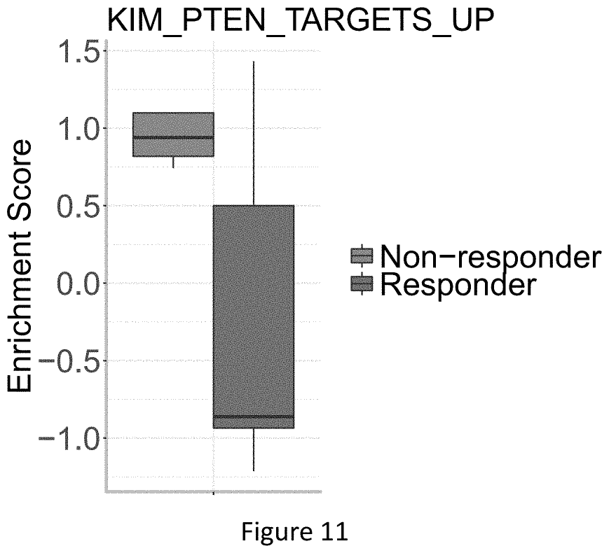

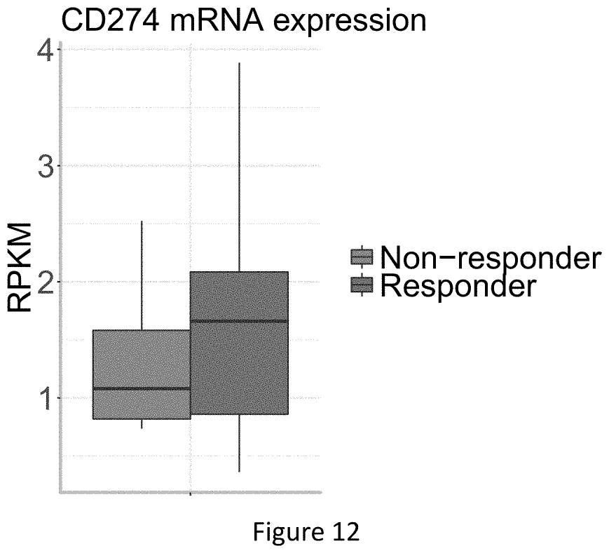

PTEN loss in tumor cells can increase the expression of immunosuppressive cytokines, resulting in decreased T-cell infiltration in tumors and inhibited autophagy, which can decrease T-cell-mediated cell death. Tumor-specific T-cells can lyse PTEN wild type glioma cells more efficiently than those expressing mutant PTEN. By utilizing single-sample gene set enrichment analysis (ssGSEA) to calculate the enrichment score of the PI3K-AKT pathway for each tumor in our cohort, significantly higher PI3K-AKT pathway activity was observed among PTEN mutant non-responsive tumors (t-test p=0.049, FIG. 11). However, no difference in PD-L1 RNA expression between responsive and non-responsive tumors was found (t-test p=0.374, FIG. 12).

Enrichment of MAPK (ERK) pathway mutations in Anti-PD-1 Responsive GBM: 4 mutations were found in the MAPK pathway components (including BRAF and PTPN11) among the 10 responders, but none among the 19 non-responders (FIG. 4B). Considering the rarity of MAPK mutations among IDH1 wild-type glioblastoma (mutation rate 7.8%, 36 of 458 tumors from TCGA), MAPK was more frequently mutated in the responsive tumors than expected (Fisher p=0.0066, odds ratio=7.74). Given the high prevalence of BRAF mutation in melanoma and the dramatic success of immunotherapy in treating advanced melanoma, this finding can have implications for the MAP kinase pathway and immune response. Moreover, the MAPK pathway was implicated in the modulation of T-cell recognition of melanoma cells in a genome-wide CRISPR screen analysis, functionally implicating these alterations in tumor cell recognition by cytotoxic T-cells, a necessary component for effective PD1-inhibitor therapy.

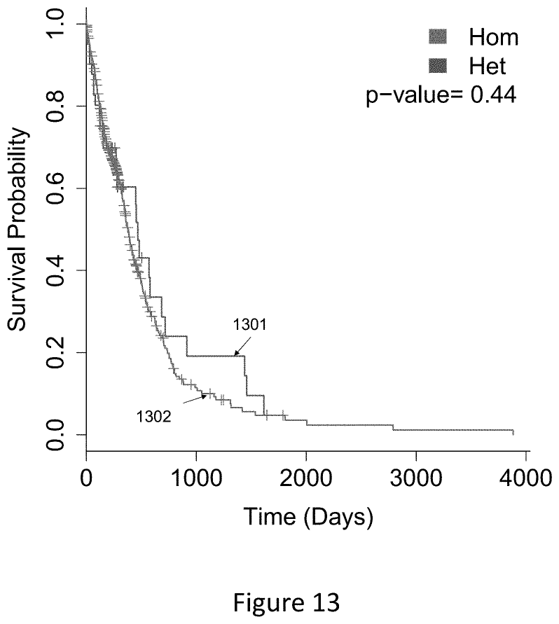

HLA heterozygosity correlates with Anti-PD-1 Response: zygosity at HLA-I genes can influence the survival of advanced melanoma and advanced non-small cell lung cancer (NSCLC) cancer patients treated with immune checkpoint blockade therapies. “PolySolver” was used to determine HLA genotypes of 15 patients for which there was normal blood whole-exome sequencing data. 5 of the 7 non-responders were homozygous for at least one HLA-I locus (A, B or C), but this was only the case for 2 of the 8 responders (Fisher p=0.131). Using the 394 GBM patients from TCGA as a background, the ratio of homozygosity 1301 at HLA-I loci was 117/394, which can lead to the statistical conclusion that HLA-I homozygosity 1302 is enriched in non-responders (Fisher p=0.029). Meanwhile, in the absence of immunotherapy, HLA zygosity does not significantly affect GBM survival (Log-rank p=0.84, FIG. 13).

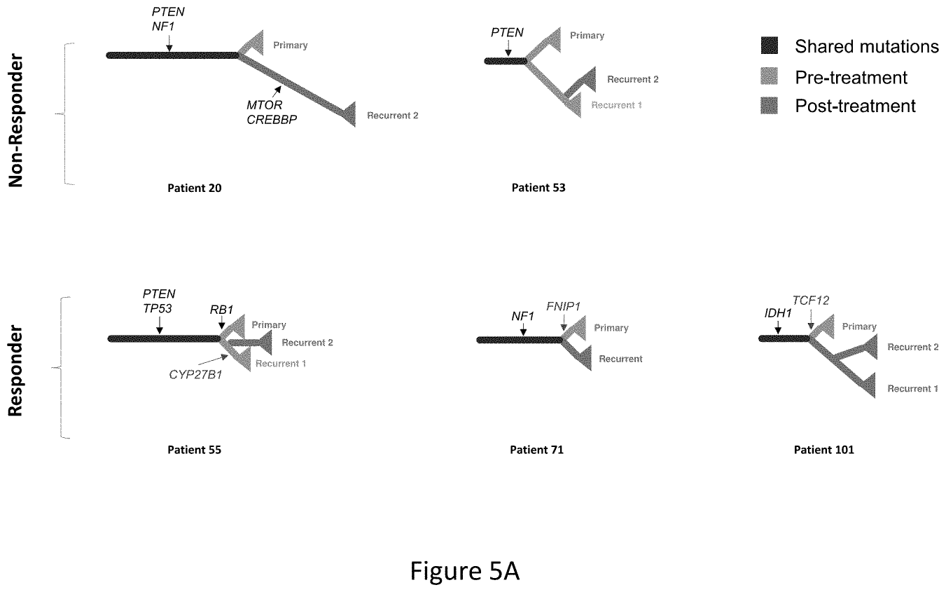

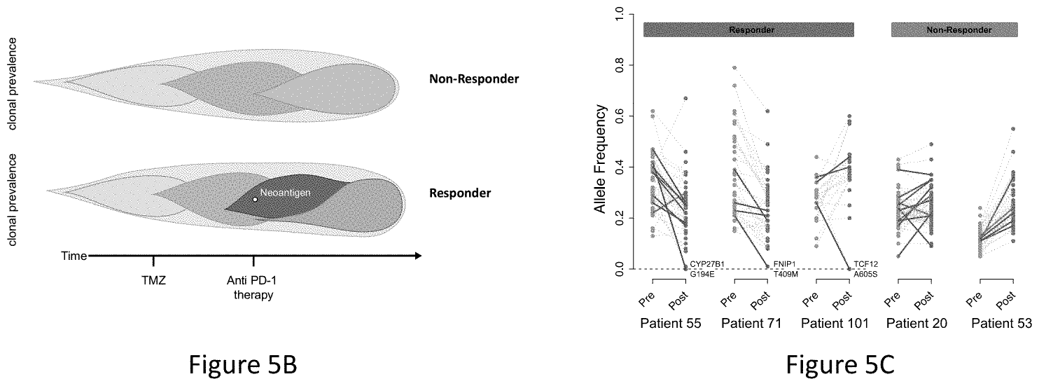

Clonal evolution of tumors under immunotherapy can reflect negative selection against neoantigens. The immune system can promote or select tumor variants with reduced immunogenicity, thereby providing developing tumors with a mechanism to escape immunologic detection and elimination. This can contribute to immune-evasive features of gliomas. The pattern of initial response and later relapse among the responders was related to the evolution of tumors undergoing anti-PD-1 immunotherapy. The number of mutations exclusive to or in common with each sample was used to construct evolutionary trees for 5 patients (2 non-responders and 3 responders) who provided both pre- and post-immunotherapy tumor samples (FIG. 5A).

The tumors from non-responders and responders exhibited different patterns of evolution. The higher fraction of mutations exclusive to post-immunotherapy tumors compared to the pre-anti-PD-1 treatment case in the two non-responders (patient 20 & 53) suggests that they followed the classical linear model of tumor evolution. In contrast, the tumors from two responders (patients 55 & 71) were more similar to the branched model, with clonal alterations in pre-anti-PD-1 dominant clone not present after therapy, suggesting that specific alterations and evolutionary patterns are associated with treatment (FIG. 5B).

In the case of Patient 55, anti-PD-1 therapy was started between the 2nd and 3rd surgery (labeled Recurrent 1 and Recurrent 2). After comparing the mutational profiles of these two samples, 3 missense mutations (MYPN R409H, UBQLN3 R159W, CYP27B1 G194E) were identified that were present in Recurrent 1, but not in Recurrent 2. Only one of these mutations (CYP27B1 G194E) is highly expressed (RPKM>5) and predicted to result in immunogenic neoantigens. For Patient 71, 1 missense mutation (FNIP1 T409M) missing was found after immunotherapy, which is also highly expressed (RPKM>5) and is predicted to generate a neoantigen. Another responder, patient 101, had IDH1 mutant GBM and received anti-PD-1 therapy after their 1st surgery. Again, only one mutation (TCF12 A605S) in the primary sample was found missing in the recurrent samples, again highly expressed (RPKM>5) and predicted to generate a neoantigen (FIG. 5C).

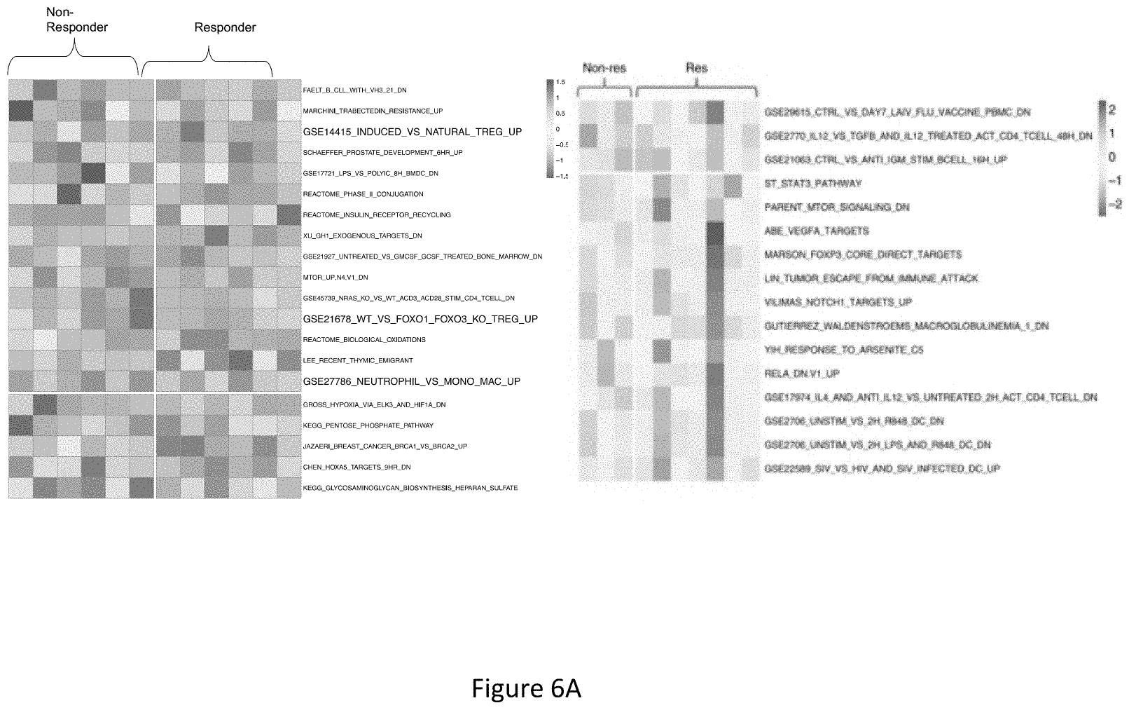

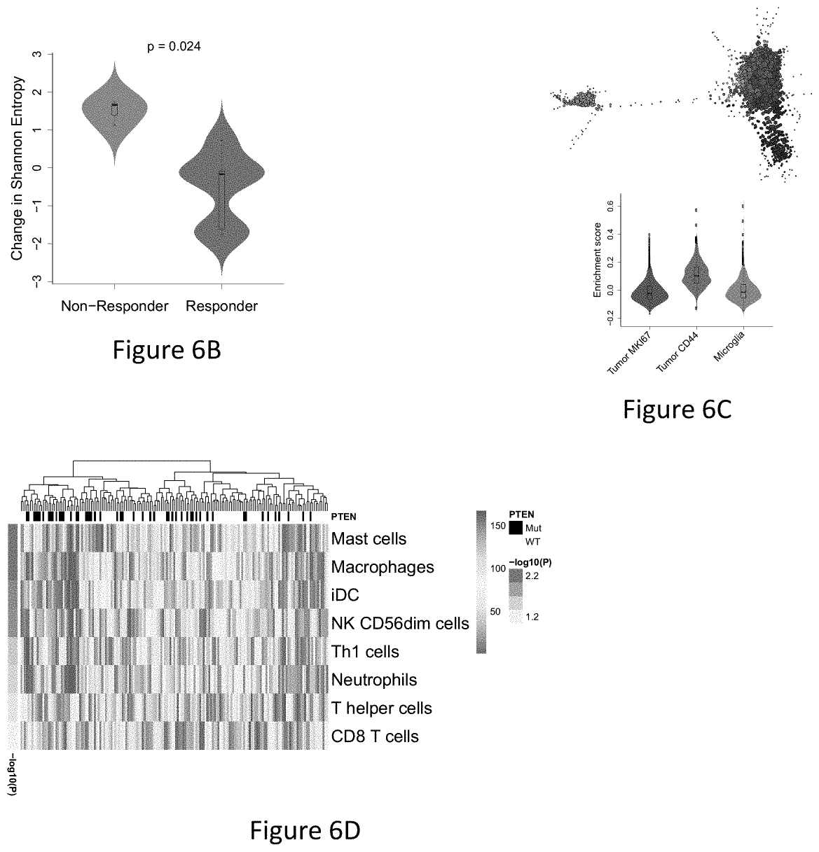

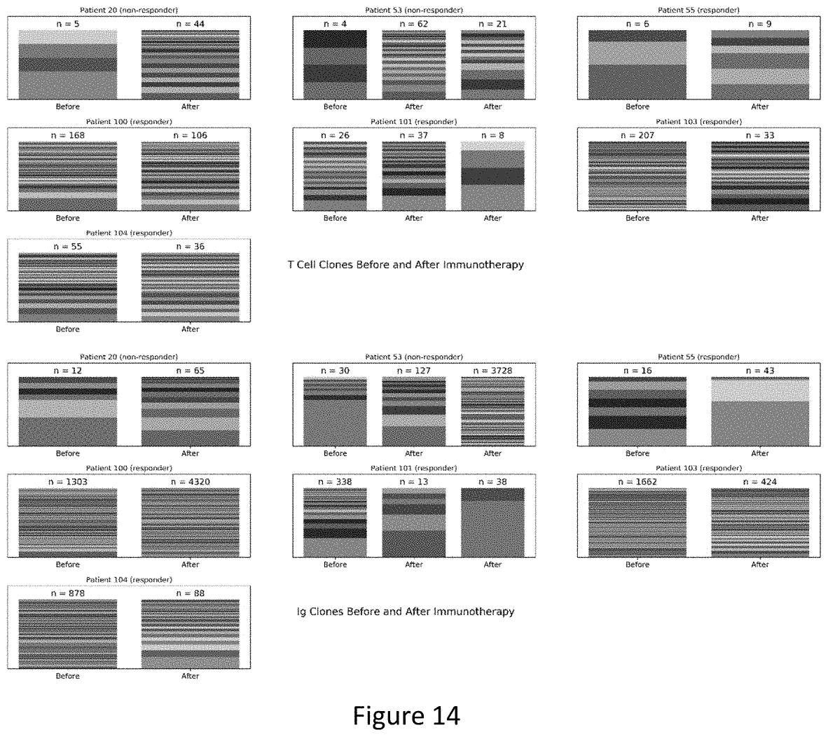

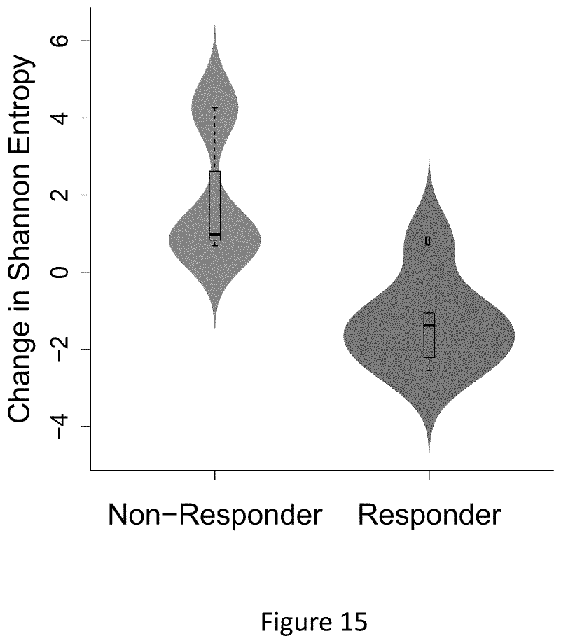

Clonal evolution of lymphocytes before and after immunotherapy: lymphocyte clonal diversity was assessed by identifying TCR and immunoglobulin RNA sequences via MiXCR. This was performed for a total of 7 patients for whom there was pre- and post-immunotherapy RNA-seq data of sufficient quality. Of these patients (2 non-responders and 5 responders), one from each response criteria had two samples post-therapy (patients 53 and 101). The total number of reads, as well as the clonal diversity via Shannon entropy, an information theory measure of randomness was assessed (FIG. 14). Non-responders had a greater increase in clonal diversity among T cells compared to responders (FIG. 6B, p=0.024, Exact Mann-Whitney U test). Likewise, the same effect was seen in the clonality of immunoglobulin reads, suggesting a similar response in B cells (FIG. 15, p=0.048, Exact Mann-Whitney U test).

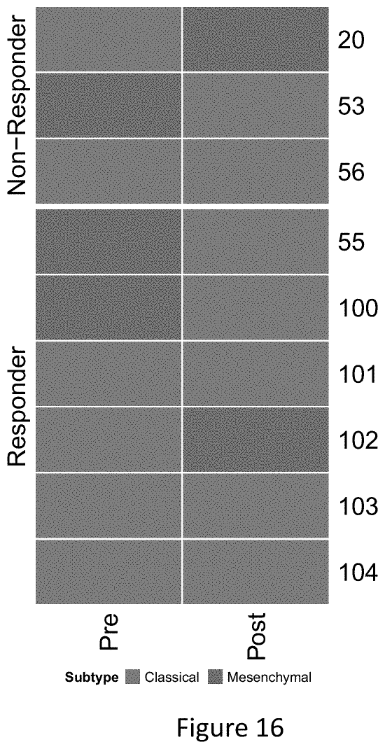

Enriched Transcriptomic Signatures in Anti-PD-1 Non-Responsive GBM: expression subtyping into proneural, mesenchymal, and classical subtypes did not result in any association with the response (FIG. 16). The transcriptomic profiles of two tumor groups were compared using ssGSEA based on 5 collections of annotated gene sets from the Molecular Signature Database v6.0 (C2 curated gene sets, C4 computational gene sets, C6 oncogenic gene sets, and C7 immunologic gene sets).

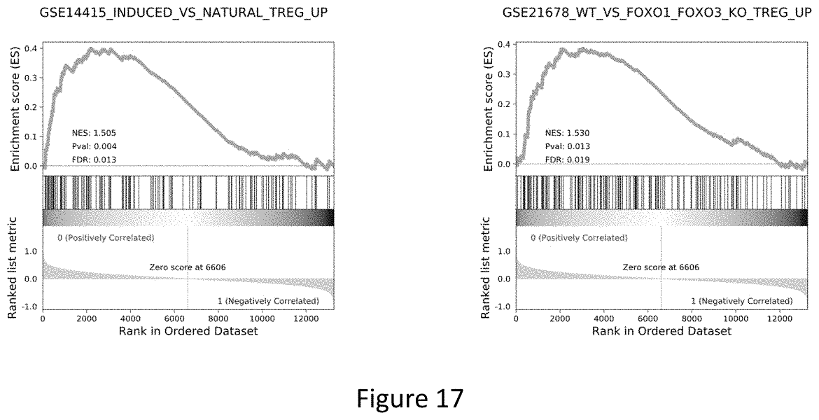

From the differential enrichment analysis across a total of 9,292 gene sets, prior to PD-1 inhibitor treatment, the gene sets related to the regulatory T cell transcription factor FOXP3 were among the top-ranked gene sets (FIG. 6A). Additionally, enrichment analysis showed that genes up-regulated in Treg cells were significantly more active in non-responsive tumors (FIG. 17). FOXP3-expressing Treg cells, which suppress the aberrant immune response against self-antigens, was negatively associated with clinical response to adoptive immunotherapy in human cancers. Following immunotherapy, gene sets related to immunosuppression were more active in responsive tumors, including FOXP3 and STAT3 signatures as well as an immune evasion signature previously reported in renal cell carcinoma (FIG. 6A).

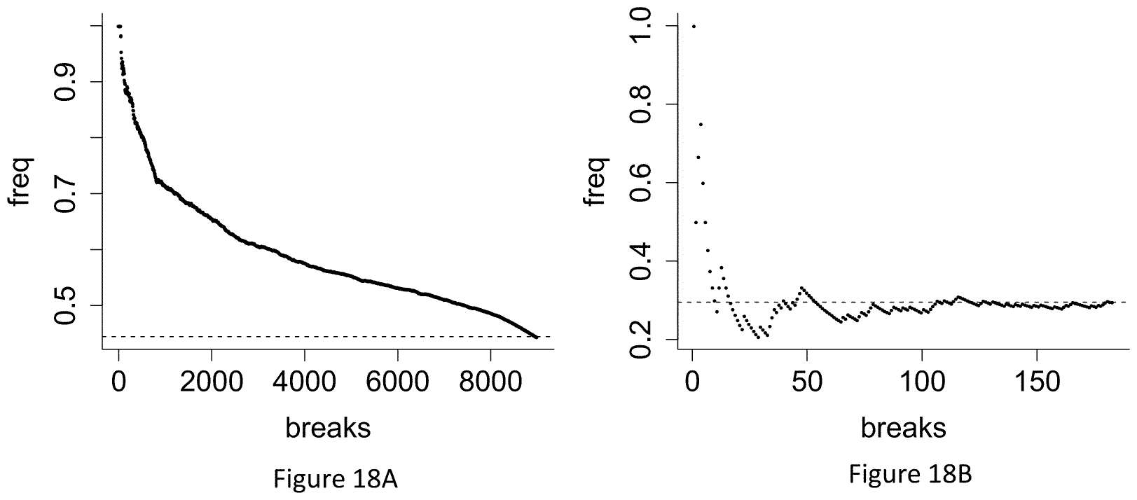

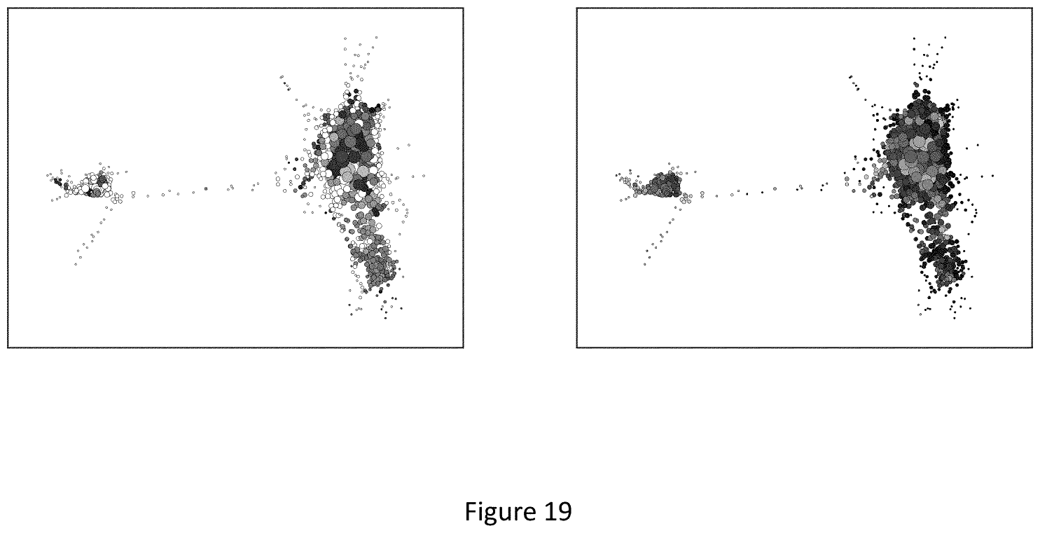

Immunohistochemistry imaging of five tumors after anti-PD-1 treatment (2 responders and 3 non-responders) did not identify CD4+FOXP3+ regulatory T-cells, indicating that the FOXP3 expression signature was not generated by these cells. To identify the origin of this immune signature, the transcriptional profiles of 9,000 cells from three GBMs including a PTEN-mutated tumor were evaluated. Cells associated with the signature were enriched in a PTEN-mutated tumor (p<1e−16, Kolmogorov-Smirnov test, FIG. 18), consistent with associations found in TCGA PTEN-mutated samples (p<1e−16, Kolmogorov-Smirnov test, FIG. 18). Using topological data analysis, three cellular populations were identified: microglia, actively proliferating tumor cells (Ki67+), and tumor cells with migrational markers (CD44+). Of these groups, the immunosuppressive signature was most associated with the CD44+ tumor subpopulation of the PTEN-mutated case (p<1e−16, t-test, FIG. 6C, FIG. 19).

The observation that immune signatures are up-regulated in non-responsive tumors compared to their responsive counterparts suggests differences between the tumor microenvironments of the two groups, indicating that PTEN can play a role in the formation of the tumor immune microenvironment. To further explore the impact of PTEN mutations on the immunophenotype of GBM, RNA-seq data from 172 samples from TCGA was evaluated. Tumor purity was estimated using the ESTIMATE algorithm.

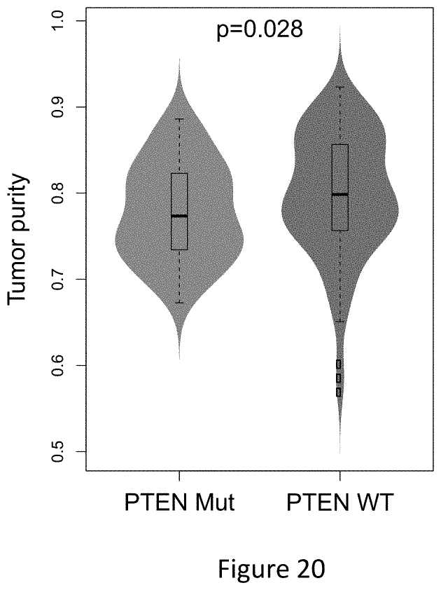

Comparing PTEN-mutated samples with PTEN wild-type samples, PTEN mutation was correlated with the aforementioned FOXP3-related transcriptional signature, and with lower tumor purity (p=0.028, Wilcoxon rank test) (FIG. 20). Then, ssGSEA was employed to measure the per sample infiltration levels of 24 immune cell types. Correlation analysis revealed that PTEN mutations can be associated with a higher level of macrophages, microglia, and neutrophils in the tumor microenvironment (FIG. 6D). As the predominant immune cells infiltrating gliomas, tumor-associated macrophages (TAMs) released a wide array of growth factors and cytokines in response to factors produced by cancer cells. TAMs can also facilitate tumor proliferation, survival, and migration. This can impact PTEN mutated patient response to anti-PD-1 therapy.

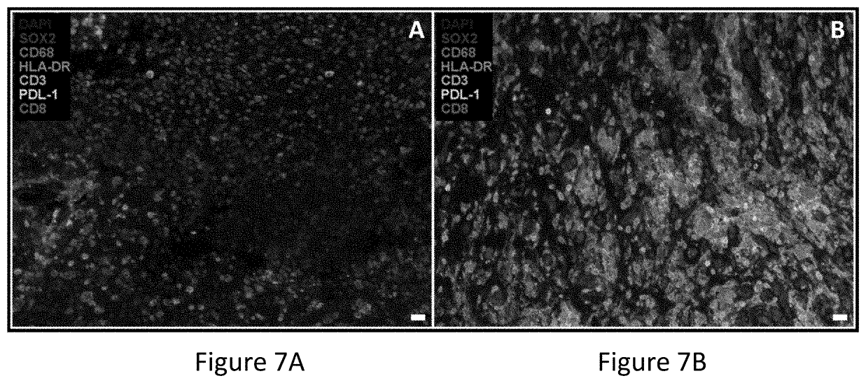

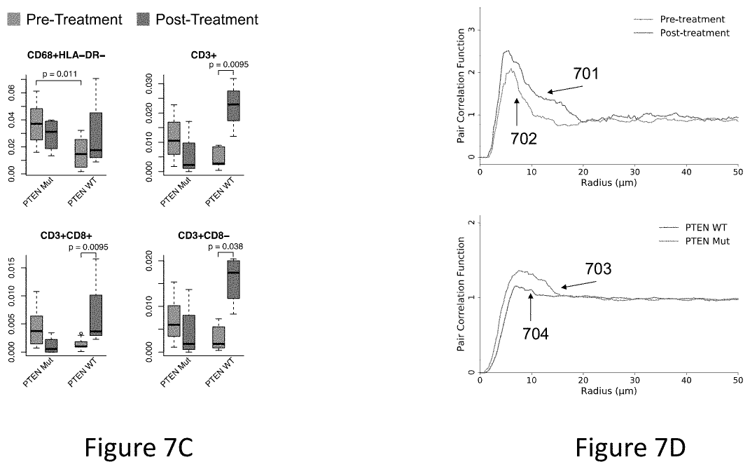

Immune Cell Infiltration Correlates with Response to anti-PD-1: PTEN mutations were examined to identify the association with the structure of the tumor microenvironment. In order to evaluate the immune cell densities, quantitative multiplex immunofluorescence (qmIF) was used to profile the tumor microenvironment of the samples in our cohort. FFPE specimens from 17 patients with matched pre- and post- anti-PD1 treatment samples were stained and analyzed (7 non-responders, 10 responders, FIGS. 7A-B). PTEN-mutated tumors tended to have higher overall levels of CD68+ macrophage infiltration, although the difference did not reach statistical significance. Furthermore, in PTEN-mutated tumors, the significantly higher density of CD68+HLA-DR− macrophages was observed (p=0.011, Wilcoxon rank-sum test, FIG. 7C), a subpopulation that indicates poor survival in melanoma. Finally, after immunotherapy, the density of CD3+ T cells in PTEN-wildtype samples significantly increased compared to pre-treatment samples (p=0.0095, Wilcoxon rank-sum test), while the PTEN-mutated samples did not show this change. The same pattern was also observed in both CD3+CD8−(p=0.0095, Wilcoxon rank-sum test, FIG. 7C) and CD3+CD8+ T cells (p=0.038, Wilcoxon rank-sum test, FIG. 7C).

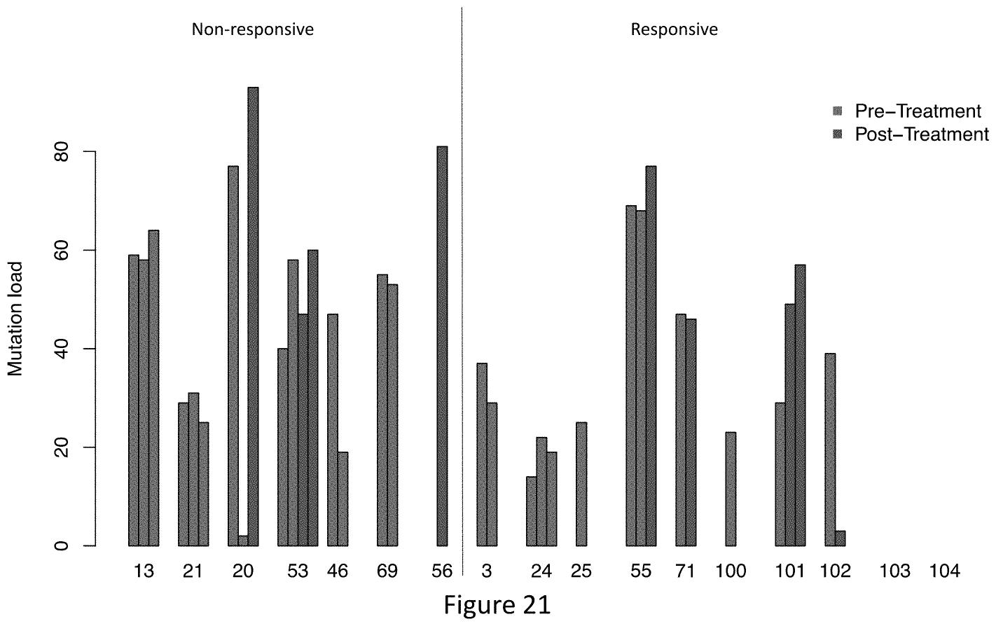

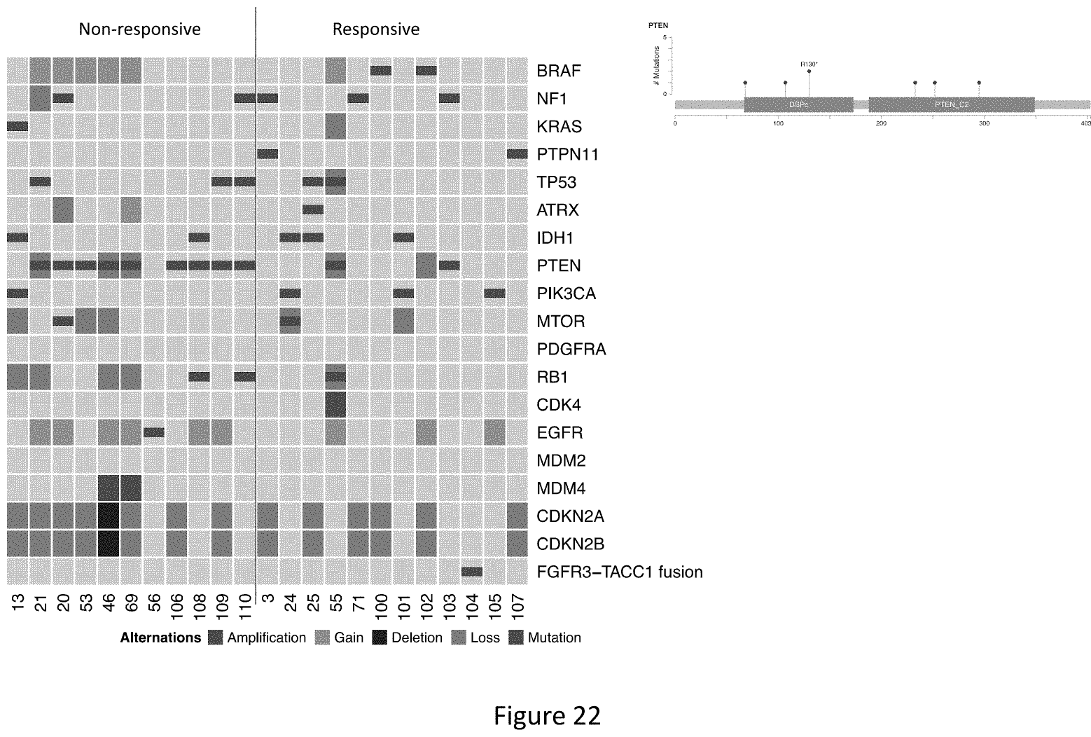

To assesses the degree of clustering between cell types, the pair correlation function, a spatial statistic technique, was used. Prior to immunotherapy 702, tumor cells clustered more strongly with each other in PTEN-mutated cases 703 compared to PTEN-wildtype 704 (p=2.4e−4, Wilcoxon rank-sum test, FIG. 7D). Furthermore, in PTEN-wildtype cases 704, macrophages strongly clustered with each other following treatment 701 (p=0.0012, Wilcoxon rank-sum test, FIG. 7D); however, this effect was reversed in PTEN mutants 703 (p=0.032). FIG. 21 shows changes in mutation loads of non-responsive and responsive patients before and after treatment. FIG. 22 shows alternations of genes in Responders and Non-Responders. Table 1 shows clinical variables ranked by the cox survival regression model.

TABLE 1
Ranking of clinical variables by Cox survival regression.

| | Survival Cox |
|---|---|
| | Regression |
| Clinical variable (+ PTEN and IDH1 mutation status) | p-value |
| Response. | 0.00093 |
| IDH1_mutation | 0.88 |
| PTEN_mutation | 0.0081 |
| Age.started.PD1.Inhibitor | 0.22 |
| Gender | 0.18 |
| Tumor.hemisphere | 0.043 |
| Tumor.Location | 0.71 |
| KPS . . . 70.1 . . . 70 . . . 0 | 0.83 |
| KPS.at.PD1.Inhibitor.start | 0.84 |
| PD1.inhibitor | 0.02 |
| Steroids.when.started.PD1.Inhibitor . . . Yes.No. | 0.92 |
| Steroids.during.PD1.Inhibitor . . . Yes.No. | 0.59 |
| Bev.failure.before.PD1.Inhibitor . . . yes.no. | 0.23 |
| First.recurrence . . . 0 | 0.19 |
| X . . . of.prior.recurrences | 0.5 |
| PD1.Inhibitor.concurrently.with.bev . . . yes.no. | 0.49 |
| PD1.Inhibitor.concurrently.with.BCNU.CCNU . . . yes.no. | 0.78 |
| PD1.Inhibitor.concurrently.with.temodar . . . yes.no. | 0.2 |
| PD1.Inhibitor.concurrently.with.Novo.TTF. | 0.67 |
| PD1.Inhibitor.concurrently.with.DC.Vax. | 0.29 |
| PD1.Inhibitor.with.imatinib. | 0.67 |
| PD1.Inhibitor.with.celecoxib. | 0.059 |
| PD1.Inhibitor.concurrently.with.re.irradiation. | 0.68 |

After analyzing the p-value for the clinical variables, as shown in Table 1, Response, PTEN mutation, Tumor hemisphere, and PD1.inhibitor (nivolumab vs perbrolizumab) showed standalone significance. Table 2 shows mean cox regression by overall survival month post-PD1 inhibitor treatment. Table 3 shows cox regression by overall survival month post-left and right tumor hemisphere treatment.

TABLE 2
Mean survival after PD.1 inhibitor treatment.

| | Nivolumab mean | Pembrolizumab mean | T-test |
|---|---|---|---|
| | survival | survival | p-value |
| PD1.inhibitor | 10.46667 | 17.94 | 0.01403 |

TABLE 3
Mean survival after Tumor hemisphere treatment.

| | Left | Right | T-test p-value |
|---|---|---|---|
| Tumor | 14.05385 | 9.54 | 0.0578 |
| hemisphere | | | |

In summary, the GBM patients who responded to anti-PD-1 immunotherapy (as evaluated by objective tumor response) had improved overall survival after treatment. Tumors from non-responders were enriched for PTEN mutations. Furthermore, RNA-seq analysis indicated that PTEN mutations can induce a distinct immunosuppressive microenvironment in tumors compared to their PTEN-wildtype counterparts. Single-cell RNA profiling revealed that the source of this signature originates not from Treg cells, but rather from tumor cells overexpressing CD44, a marker associated with cellular mobility and GBM aggressiveness. Immunohistochemistry analysis confirmed the lack of increase of T-cell infiltration in PTEN mutant tumors and an associated increased macrophage population. Differences were identified in the spatial structure of the tumor microenvironment that was associated with PTEN status; the increased clustering of tumor cells in PTEN mutants can impede immune infiltration. Similar results in melanoma, where PTEN loss was found to be associated with resistance to immune infiltration, showed that PTEN immunosuppressive effects were related to the production of inhibitory cytokines and reduced autophagic activity leading to T-cell induced tumor apoptosis. Furthermore, the AKT-mTOR pathway downstream of PTEN was implicated in both PD-L1 expressions as well as immune evasion in cancers. These phenotypes can determine the response pattern of GBM patients to anti-PD-1 immunotherapy.

Alterations in the MAPK signaling pathway can be implicated in the development of an unfavorable cancer immune phenotype. MAPK pathway inhibition can also increase the efficacy of immunotherapy. The observed BRAF/PTPN11 mutations were able to be enriched in tumors responding to anti-PD-1 therapy suggesting the strategy of combining checkpoint inhibitors with MAPK targeted therapy in multiple cancers.

The distinct evolutionary patterns of responding tumors and non-responding tumors under immunotherapy were identified. The analysis of their evolution provides evidence that the immune system can play an important role in the negative selection of clones containing immunogenic neoepitopes, and thus promote tumors in escaping immune surveillance. For example, low mutational loads did not preclude tumor infiltration by mutation-reactive, class I- and II-restricted T-cells in gastrointestinal cancers. Different patterns in the changes of the clonal composition of lymphocytes were identified between responders and non-responders. Non-responders were found to have a greater increase in T-cell diversity following immunotherapy, suggesting the failure of selective recruitment of lymphocytes into the tumor microenvironment. Supporting the role of tumor evolution in shaping the microenvironment, gene sets associated with immunosuppression were more active in non-responders prior to immunotherapy but were more active in responders following treatment. Tumors of non-responders possessed primary resistance to immunotherapy, whereas responders demonstrate a gradual acquisition of resistance following successful selection pressure.

Whereas overall, PD-1 inhibitors do not provide a survival benefit for GBM patients, a sub-group of patients can benefit from this therapy. Moreover, molecular profiling of responders and non-responders can inform a personalized approach and refine patient selection for immunotherapy. The disclosed techniques can provide techniques for the effective application of therapy for GBM, cancer that is notorious for its molecular heterogeneity. In conclusion, multiple genomic features related to response to anti-PD-1 therapy in GBM patients were identified. Distinct evolutionary patterns of GBM were also observed under immunotherapy. The disclosed techniques can inform therapeutic options and identify mechanisms of resistance to immunotherapies in these tumors.

In addition to the various embodiments depicted and claimed, the disclosed subject matter is also directed to other embodiments having other combinations of the features disclosed and claimed herein. As such, the particular features presented herein can be combined with each other in other manners within the scope of the disclosed subject matter such that the disclosed subject matter includes any suitable combination of the features disclosed herein.

The foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the methods and systems of the disclosed subject matter without departing from the spirit or scope of the disclosed subject matter. Thus, it is intended that the disclosed subject matter include modifications and variations that are within the scope of the appended claims and their equivalents.

What is claimed is:

1. A method for predicting a sensitivity of the cancer to an anti-programed cell death 1 (PD-1) immunotherapy, comprising:
in a sample of the cancer, determining a presence of at least one mutation in at least one target gene, wherein the target gene/protein includes a PTEN, a PTPN11, and/or a BRAF gene/protein,
where if the PTPN11 or BRAF gene/protein includes at least one mutation and/or the PTEN gene/protein is a wild type PTEN/protein gene, then the cancer is predicted to be sensitive to the PD-1 immunotherapy.

2. The method of claim 1, further comprising determining a presence of at least mutation in MAPK/ERK pathway components in the sample.

3. The method of claim 1, further comprising determining a PI3K-AKT pathway activity level of the sample.

4. The method of claim 1, further comprising determining a heterozygosity of human leukocyte antigen (HLA) in the sample.

5. The method of claim 1, further comprising identifying clustering of cancer cells and immune cell infiltration, wherein immune cell includes lymphocytes, neutrophils, macrophages, monocytes, or combinations thereof.

6. The method of claim 1, further comprising identifying transcriptomic signatures, wherein transcriptomic signatures include an immune evasion, a FOXP3 expression, a STAT3 expression, an immunosuppressive response, or combinations thereof.

7. The method of claim 1, further comprising providing the cancer with a PD-1 inhibitor.

8. The method of claim 7, further comprising identifying a clonal evolution of the cancer before and after the PD-1 inhibitor treatment.

9. A kit for predicting a sensitivity of a cancer to an anti-programed cell death 1 (PD-1) immunotherapy, comprising
a system comprising:
one or more processors; and
one or more computer-readable non-transitory storage media coupled to one or more of the processors and comprising instructions operable when executed by one or more of the processors to cause the system to
determine a presence of at least one mutation in at least one target gene from a sample of the cancer,
wherein if the target gene includes a PTEN, a PTPN11, and/or a BRAF gene, and where if the PTPN11 or BRAF gene includes at least one mutation and/or the PTEN gene is a wild type PTEN gene, then the cancer is predicted to be sensitive to the PD-1 immunotherapy.

10. The kit of claim 9, wherein the kit is adapted to determine a presence of at least mutation in MAPK/ERK pathway components in the sample.

11. The kit of claim 9, wherein the kit is adapted to determine a PI3K-AKT pathway activity level of the sample.

12. The kit of claim 9, wherein the kit is adapted to determine a heterozygosity of human leukocyte antigen (HLA) in the sample.

13. The kit of claim 9, wherein the kit is adapted to identify clustering of cancer cells and immune cell infiltration, wherein immune cell includes lymphocytes, neutrophils, macrophages, monocytes, or combinations thereof.

14. The kit of claim 9, wherein the kit is adapted to identify transcriptomic signatures, wherein transcriptomic signatures include an immune evasion, a FOXP3 expression, a STAT3 expression, an immunosuppressive response, or combinations thereof.

15. The kit of claim 9, further comprising a PD-1 inhibitor, wherein the PD-1 inhibitor comprises pembrolizumab, nivolumab, or a combination thereof.

16. The kit of claim 9, wherein the kit is adapted to identify a clonal evolution of the cancer before and after a PD-1 inhibitor treatment.
